(19) United States
(12) Patent Application Publication
(10) Pub. No.: US 2021/0386670 A1
(43) Pub. Date: Dec. 16, 2021

(54) NANOEMULSION SYSTEM FOR TRANSDERMAL DELIVERY OF PHARMACEUTICAL COMPOSITIONS AND OTHER ACTIVE AGENTS

(71) Applicant: SERGIO BEZERRA DE MENEZES, MIAMI, FL (US)

(72) Inventor: SERGIO BEZERRA DE MENEZES, MIAMI, FL (US)

(21) Appl. No.: 16/899,219

(22) Filed: Jun. 11, 2020

(51) Int. Cl. A61K 9/107; A61K 9/00; A61K 47/14; A61K 47/10; A61K 47/26; A61K 47/20; A61K 47/18; A61K 47/12; A61K 36/355

(52) U.S. Cl. CPC A61K 9/107; A61K 9/0014; A61K 47/14; A61K 47/10; A61K 45/06; A61K 47/20; A61K 47/183; A61K 47/12; A61K 36/355; A61K 47/26

(57) ABSTRACT

A nanoemulsion system for transdermal delivery of a pharmaceutical composition or other active agent across at least an outer layer of skin of a patient includes a first phase, a second phase, and a third phase, and at least one pharmaceutical composition or other active agent. The first phase comprises an oil phase, the second phase comprises an aqueous phase, and the third phase comprises a final phase including a preservation agent. A method of preparing a nanoemulsion system for transdermal delivery of at least one pharmaceutical composition or other active agent across at least an outer layer of skin of a patient includes preparing predetermined amounts of first, second and third phases, heating and combining the first and second phases while stirring, cooling the initial mixture prior to adding the third phase, and cooling the resultant mixture to a final temperature while stirring to form the nanoemulsion system.

### BACKGROUND OF THE INVENTION

### Field of the Invention

The present invention is directed to a nanoemulsion system for transdermal delivery of at least one pharmaceutical composition or other active agent across at least an outer layer of skin of a patient. The present invention is further directed to a method of preparing a nanoemulsion system for transdermal delivery of at least one pharmaceutical composition or other active agent across at least an outer layer of skin of a patient.

### Description of the Related Art

The transdermal pathway is an efficient pathway for the administration of pharmaceutical compositions, acting as an alternative to oral and injectable administration, especially when use of the gastrointestinal tract is contraindicated. This pathway can be used to administer pharmaceutical compositions and/or other active agents from various therapeutic classes, for example, anti-inflammatory, corticosteroids and hormones, just to name a few. This pathway allows control of the absorption of a certain amount of pharmaceutical composition or other active agent, it permits application to different specific locations of the body, it can increase patient adherence to treatment due to ease of administration and decreased systemic toxicity and side effects.

The considerable challenge posed by the transdermal route of administration is that the skin is a barrier that is very difficult to permeate. Thus, without the use of appropriate technologies, it is difficult for a pharmaceutical composition or other active agent applied topically to the skin to be made available in the bloodstream for systemic action.

Conventional gels and emulsions are usually produced by dispersing polymers in water and fragmenting an oil phase into an aqueous phase in the presence of a surfactant. These systems comprise a high particle size, and thus, it is difficult for them to permeate through the stratum corneum. These systems are not efficient administration of pharmaceutical compositions and/or other active agents to a patient via a transdermal administration route.

Nanoemulsions have a particle size which is much better suited for permeation through the stratum corneum, and the negative Zeta Potential of nanoemulsions enable the attraction of pharmaceutical compositions and active agents into the deeper layers of the skin. More in particular, the Zeta Potential may be defined as a measure of the magnitude of repulsion or electrostatic attraction or charge between particles. It is a fundamental parameter for evaluating a nanostructured system. If a formulation is stable and nanostructured, it will have a negative Zeta Potential value within the established parameters.

Some nanoemulsions comprise chemical preservatives, polyethylene glycols and silicones. Such nanoemulsions may hinder a compatibility with the stratum corneum further leading way to detracting from efficient delivery of pharmaceuticals and/or other active agents.

Thus, it would be beneficial to provide a nanoemulsion system for transdermal delivery which is a biomimetic formulation, i.e., a nanoemulsion which contains biologically identical ingredients to the skin components or are otherwise substantially compatible with it. A further advantage may be realized by providing a nanoemulsion system for transdermal delivery comprising a stable formulation with a small particle size, i.e., on the nanometer scale, comprised of dermato-compatible ingredients that allow the permeation of pharmaceutical compositions and/or other active agents gradually and continuously into, through and across the stratum corneum.

Another benefit which may be realized by utilizing a nanoemulsion system for transdermal delivery is the formation of liquid crystals. More in particular, liquid crystals are formed by lamellar bilayers that give nanoemulsions extra-hydrating power by mimicking the lipid bilayers of the cell membrane.

Still a further benefit of providing a nanoemulsion system for transdermal delivery is that Brownian motion will narrow and verify particle size distribution, and Raman Confocal Spectroscopy may be utilized to verify the permeation and gradual release of active agents conveyed via a nanoemulsion system for transdermal delivery. It would also be beneficial to provide a stable nanoemulsion having a biocompatible Zeta Potential of about −40 millivolts to about −60 millivolts.

Numerous additional benefits may be realized from a nanoemulsion system for transdermal delivery of pharmaceutical compositions and/or other active agents which include, but not limited to: absence of pruritus, skin irritation, pain in removal and aesthetic problems very common in other transdermal use systems, such as patches or patches; excellent biocompatibility; verifiable permeation and gradual release via Confocal Raman Spectroscopy; minimization of the frequency of administration per day; maintenance of the therapeutic index of the pharmaceutical composition and/or active agent; avoidance of first pass metabolism in the liver; absence of unpleasant taste, common in orally administered pharmaceutical compositions and/or active agents; easily adaptable to accommodate changes in dosage; easy administration of pharmaceutical compositions and/or active agents through the skin; increased patient compliance with therapy; high power of skin hydration; and particle sizes in the nanometer scale.

### SUMMARY OF THE INVENTION

As before, in at least one embodiment, the present invention is directed to a nanoemulsion system for transdermal delivery of at least one pharmaceutical composition or other active agent across at least an outer layer of skin of a patient which includes a first phase, a second phase, a third phase, and the at least one pharmaceutical composition or other active agent.

In one embodiment, a first phase of a nanoemulsion system in accordance with the present invention comprises an oil phase. More in particular, a first phase of a nanoemulsion system comprises an amount of at least one of cetearyl olivate, sorbitan olivate, cetearyl alcohol, polysorbate 60, cetearyl glucoside, or olive oil PEG-7 esters. In yet another embodiment of a nanoemulsion system in accordance with the present invention, a first phase comprises an amount of at least one of copernicia cerifera wax, cetyl acetate, stearyl acetate, oleyl acetate, acetylated lanolin alcohols, butylated hydroxytoluene (BHT), tocopheryl acetate, or benzoic acid. The first phase of a nanoemulsion system in accordance with still one further additional embodiment of the present invention comprises an amount of at least one of Vitis vinifera seed oil, Linum usitatissimum seed oil, Prunus amygdalus dulcis oil, Persea gratissima oil, or jojoba oil.

In accordance with at least one further embodiment of the present invention, a second phase comprises an aqueous phase which comprises an amount of water. In one further embodiment, a second phase comprises an amount of at least one of propylene glycol, disodium EDTA, sodium benzoate, or potassium sorbate.

As previously disclosed, a nanoemulsion system in accordance with one embodiment of the present invention also comprises a third, or final phase. In accordance with at least one embodiment, a final phase comprises an amount of at least one of a preservation agent or a permeation promoter. In at least one embodiment, a final phase of a nanoemulsion system in accordance with the present invention comprises an amount of at least one of pentylene glycol, glycerin, sodium lactate, lactic acid, serina, urea, sorbitol, sodium chloride, allantoin, citric acid, dimethyl sulfoxide, or water. In yet another embodiment, a final phase of a nanoemulsion system in accordance with the present invention comprises an amount of at least one of ethoxydiglycol diethylene glycol monoethyl ether or dimethyl sulfoxide. A final phase of a nanoemulsion system in accordance with another embodiment of the present invention further comprises a preservation agent.

The present invention is further directed to a method of preparing a nanoemulsion system for transdermal delivery of at least one pharmaceutical composition or other active agent across at least an outer layer of skin of a patient. In at least one embodiment, the present includes one or more of: preparing a predetermined amount of a first phase; preparing a predetermined amount of a second phase; heating the predetermined amount of the first phase to an upper threshold temperature under constant stirring; heating the predetermined amount of second phase to the upper threshold temperature under constant stirring; adding the predetermined amount of second phase at the upper threshold temperature into the predetermined amount of the first phase at the upper threshold temperature under constant stirring to form an initial mixture; cooling the initial mixture to a lower threshold temperature under constant stirring; preparing a predetermined amount of a third phase; adding the predetermined amount of the third phase to the initial mixture cooled to the lower threshold temperature under constant stirring to form a final mixture; cooling the final mixture to a final temperature under constant stirring to form the nanoemulsion system; and/or adjusting a pH of the nanoemulsion system.

These and other objects, features and advantages of the present invention will become clearer when the detailed description is taken into consideration.

### DETAILED DESCRIPTION OF THE PREFERRED EMBODIMENT

As before, the present invention is directed to a nanoemulsion system for transdermal delivery of at least one pharmaceutical composition and/or other active agent across at least an outer layer of skin of a patient.

As one example, an active agent may comprise a hormone, such as, but not limited to progesterone, testosterone, estrogen, etc. As another example, an active agent may comprise an anti-inflammatory composition including, but not limited to, diclofenac sodium, diclofenac potassium, ketoprofen, etc., just to name a few. A further example of an active agent in accordance with the present invention is a corticoid, such as, but once again, not limited to, hydrocortisone, betamethasone, etc. Active agents for use with the present invention include antibiotic compositions, such as, by way of example only, clindamycin, etc. It is also envisioned to be within the scope and intent of the present invention for an active agent to comprise an antifungal agent such as, once again, by way of example only, ketoconazole, miconazole, etc. It is further envisioned that an active agent in accordance with the present invention comprises a cosmetic composition such as vitamin C, retinol, hyaluronic acid, etc., among others. In at least one embodiment, an active agent for use with the present invention comprises a cytostatic composition including, but as with the foregoing, not limited to, tamoxifen, anastrozole, etc. In at least another embodiment, an active agent with the present invention may comprise a cannabidiol, dronabinol, delta-9-tetrahydrocannabinol, tetrahydrocannabinol, and/or sub products of cannabis such as, but not limited to, terpenes and/or flavonoids. It will be appreciated by those of skill in the art the foregoing is merely a partial exemplary listing of the myriad of pharmaceutical compositions and/or other active agents which may be administered topically to a patient utilizing the present nanoemulsion system for transdermal delivery, and that it is clearly within the scope and intent of the present invention for any of the myriad of pharmaceutical compositions and/or other active agents to be administered topically to a patient utilizing the present nanoemulsion system for transdermal delivery.

In at least one embodiment of the present invention, a nanoemulsion system for transdermal delivery of at least one pharmaceutical composition or other active agent across at least an outer layer of skin of a patient comprises a first phase, a second phase, a third phase, and at least one pharmaceutical composition or other active agent.

As one example, in at least one embodiment, at least one pharmaceutical composition or other active agent may comprise a hormone, such as, but not limited to progesterone, testosterone, estrogen, etc. As another example, an active agent may comprise an anti-inflammatory composition including, but not limited to, diclofenac sodium, diclofenac potassium, ketoprofen, etc., just to name a few. A further example of an active agent in accordance with the present invention is a corticoid, such as, but once again, not limited to, hydrocortisone, betamethasone, etc. Active agents for use with the present invention include antibiotic compositions, such as, by way of example only, clindamycin, etc. It is also envisioned to be within the scope and intent of the present invention for an active agent to comprise an antifungal agent such as, once again, by way of example only, ketoconazole, miconazole, etc. It is further envisioned that an active agent in accordance with the present invention comprises a cosmetic composition such as vitamin C, retinol, hyaluronic acid, etc., among others. In at least eon embodiment, an active agent for use with the present invention comprises a cytostatic composition including, but as with the foregoing, not limited to, tamoxifen, anastrozole, etc. It will be appreciated by those of skill in the art the foregoing is merely a partial exemplary listing of the myriad of pharmaceutical compositions and/or other active agents which may be administered topically to a patient utilizing the present nanoemulsion system for transdermal delivery, and that it is clearly within the scope and intent of the present invention for any of the myriad of pharmaceutical compositions and/or other active agents to be administered topically to a patient utilizing the present nanoemulsion system for transdermal delivery.

As previously disclosed, in one embodiment, a nanoemulsion system for transdermal delivery of at least one pharmaceutical compositions or other active agent across at least an outer layer of skin of a patient comprises a first phase. In at least one embodiment, a first phase of a nanoemulsion system in accordance with the present invention comprises an oil phase. More in particular, a first phase of a nanoemulsion system comprises an amount of at least one of cetearyl olivate, sorbitan olivate, cetearyl alcohol, polysorbate 60, cetearyl glucoside, or olive oil PEG-7 esters. In yet one further embodiment, a first phase of a nanoemulsion system comprises an amount of each of cetearyl olivate, sorbitan olivate, cetearyl alcohol, polysorbate 60, cetearyl glucoside, and olive oil PEG-7 esters.

In yet another embodiment of a nanoemulsion system in accordance with the present invention, a first phase comprises an amount of at least one of copernicia cerifera wax, cetyl acetate, stearyl acetate, oleyl acetate, acetylated lanolin alcohols, butylated hydroxytoluene (BHT), tocopheryl acetate, or benzoic acid. In at least one embodiment, a first phase of a nanoemulsion system comprises an amount of each of copernicia cerifera wax, cetyl acetate, stearyl acetate, oleyl acetate, acetylated lanolin alcohols, butylated hydroxytoluene (BHT), tocopheryl acetate, and benzoic acid.

The first phase of a nanoemulsion system in accordance with still one further additional embodiment of the present invention comprises an amount of at least one of Vitis vinifera seed oil, Linum usitatissimum seed oil, Prunus amygdalus dulcis oil, Persea gratissima oil, or jojoba oil. In yet one further additional embodiment, the first phase of a nanoemulsion system in accordance with the present invention comprises an amount of each of Vitis vinifera seed oil, Linum usitatissimum seed oil, Prunus amygdalus dulcis oil, Persea gratissima oil, and jojoba oil.

As previously disclosed, a nanoemulsion system in accordance with at least one embodiment of the present invention further comprises a second phase. In at least one embodiment, a second phase comprises an aqueous phase. More in particular, in one embodiment, a second phase of a nanoemulsion system in accordance with the present invention comprises an amount of water.

In one further embodiment, a second phase of a nanoemulsion system in accordance with the present invention comprises an amount of at least one of propylene glycol, disodium EDTA, sodium benzoate, or potassium sorbate. In at least one other embodiment of the present invention, a second phase comprises an amount of each of propylene glycol, disodium EDTA, sodium benzoate, and potassium sorbate.

Also previously disclosed, a nanoemulsion system in accordance with one embodiment of the present invention comprises a third phase. A third phase in accordance with at least one embodiment of the present invention comprises a final phase. In accordance with at least one further embodiment, a final phase comprises an amount of at least one of a preservation agent or a permeation promoter. In at least one embodiment, a final phase of a nanoemulsion system in accordance with the present invention comprises an amount of at least one of pentylene glycol, glycerin, sodium lactate, lactic acid, serina, urea, sorbitol, sodium chloride, allantoin, citric acid, dimethyl sulfoxide, or water. In at least one further embodiment, a final phase of a nanoemulsion system in accordance with the present invention comprises an amount of each of pentylene glycol, glycerin, sodium lactate, lactic acid, serina, urea, sorbitol, sodium chloride, allantoin, citric acid, dimethyl sulfoxide, and water.

In yet another embodiment, a final phase of a nanoemulsion system in accordance with the present invention comprises an amount of at least one of ethoxydiglycol diethylene glycol monoethyl ether or dimethyl sulfoxide. In at least one further embodiment, a final phase of a nanoemulsion system in accordance with the present invention comprises an amount of at least one of ethoxydiglycol diethylene glycol monoethyl ether purified or dimethyl sulfoxide. In yet one further embodiment, a final phase of a nanoemulsion system in accordance with the present invention comprises an amount of each of ethoxydiglycol diethylene glycol monoethyl ether and dimethyl sulfoxide, and in still one further embodiment, a final phase comprises an amount of each of ethoxydiglycol diethylene glycol monoethyl ether purified and dimethyl sulfoxide.

As disclosed above, a final phase of a nanoemulsion system in accordance with at least one embodiment of the present invention comprises a preservation agent. In at least one embodiment, a preservation agent comprises a natural preservative. More in particular in one embodiment of a nanoemulsion system in accordance with the present invention, a natural preservative comprises an amount of at least one of Lonicera caprifolium extract or Lonicera japonica extract in an amount of water. In yet one further embodiment, a natural preservative comprises an amount of at least one of Lonicera caprifolium extract and Lonicera japonica extract in an amount of water.

Example 1 presented below is exemplary of one formation for a nanoemulsion system for transdermal delivery of at least one pharmaceutical compositions or other active agent across at least an outer layer of skin of a patient, based on a weight percentage of each of the components specified in the formulation.

### Example 1

| Component | Weight (%) |
|---|---|
| FIRST PHASE: | |
| Cetearyl Olivate + Sorbitan Olivate | 1.0 to 10.0 |
| Cetearyl Alcohol + Polysorbate 60 + Cetearyl Glucoside | 1.0 to 5.0 |
| Olive Oil PEG-7 Esters | 1.0 to 5.0 |
| Copernicia Cerifera Wax | 0.01 to 1.0 |
| Cetyl Acetate + Stearyl Acetate + Oleyl Acetate + | 0.01 to 1.0 |
| Acetylated lanolin Alcohols | |
| Butylated Hydroxytoluene (BHT) | 0.01 to 1.0 |
| Tocopheryl Acetate | 0.01 to 1.0 |
| Benzoic Acid | 0.01 to 1.0 |
| Vitis Vinifera Seed Oil | 0.5 to 5.0 |
| Linum Usitatissimum Seed Oil | 0.5 to 5.0 |
| Prunus Amygdalus Dulcis Oil | 0.5 to 5.0 |
| Persea Gratissima Oil | 0.5 to 5.0 |
| Jojoba Oil | 0.01 to 1.0 |
| SECOND PHASE: | |
| Disodium EDTA | 0.01 to 1.0 |
| Propylene Glycol | 1.0 to 10.0 |
| Sodium Benzoate | 0.1 to 1.0 |
| Potassium Sorbate | 0.1 to 1.0 |
| Water | 60 to 80 |
| THIRD PHASE: | |
| Water + Pentylene Glycol + Glycerin + Sodium | 1.0 to 10.0 |
| Lactate + Lactic Acid + Serina + Urea + Sorbitol + | |
| Sodium Chloride + Allantoin | |
| Citric Acid | 0.01 to 1.0 |
| Dimethyl Sulfoxide | 0.1 to 5.0 |
| Ethoxydiglycol diethylene glycol monoethyl ether | 1.0 to 10.0 |
| purified | |
| Lonicera Caprifolium extract + Lonicera japonica | 0.1 to 5.0 |
| extract + Water* | |

*In at least one embodiment, the mixture of lonicera caprifolium extract + lonicera japonica extract + water is neutralized to a pH of about 6.0 utilizing an appropriate amount of citric acid. In one further embodiment, the citric acid solution has a concentration of 50 percent.

Example 2 presented below is exemplary of one further formation for a nanoemulsion system for transdermal delivery of at least one pharmaceutical compositions or other active agent across at least an outer layer of skin of a patient, once again, based on a weight percentage of each of the components specified in the formulation.

### Example 2

| Component | Weight (%) |
|---|---|
| FIRST PHASE | |
| Cetearyl Olivate + Sorbitan Olivate | 8.0 |
| Cetearyl Alcohol + Polysorbate 60 + Cetearyl Glucoside | 5.0 |
| Olive Oil PEG-7 Esters | 3.0 |
| Copernicia Cerifera Wax | 0.01 |
| Cetyl Acetate + Stearyl Acetate + Oleyl Acetate + | 0.5 |
| Acetylated lanolin Alcohols | |
| Butylated Hydroxytoluene (BHT) | 0.2 |
| Tocopheryl Acetate | 0.5 |
| Benzoic Acid | 0.2 |
| Vitis Vinifera Seed Oil | 0.5 |
| Linum Usitatissimum Seed Oil | 0.5 |
| Prunus Amygdalus Dulcis Oil | 0.5 |
| Persea Gratissima Oil | 0.5 |
| Jojoba Oil | 0.1 |
| SECOND PHASE | |
| Disodium EDTA | 0.1 |
| Propylene Glycol | 5.0 |
| Sodium Benzoate | 0.2 |
| Potassium Sorbate | 0.2 |
| Water | 70.39 |
| THIRD PHASE | |
| Water + Pentylene Glycol + Glycerin + Sodium | 2.0 |
| Lactate + Lactic Acid + Serina + Urea + Sorbitol + | |
| Sodium Chloride + Allantoin | |
| Citric Acid | 0.1 |
| Dimethyl Sulfoxide | 0.5 |
| Ethoxydiglycol diethylene glycol monoethyl ether | 1.0 |
| purified | |
| Lonicera Caprifolium extract + Lonicera japonica | 1.0 |
| extract + Water* | |
| TOTAL | 100.00% |

*In at least one embodiment, the mixture of lonicera caprifolium extract + lonicera japonica extract + water is neutralized to a pH of about 6.0 utilizing an appropriate amount of citric acid. In one further embodiment, the citric acid solution has a concentration of 50 percent.

Example 3 presented below is exemplary of a formation for 100.00 grams of a nanoemulsion system for transdermal delivery of at least one pharmaceutical compositions or other active agent across at least an outer layer of skin of a patient, including the weight, in grams, of each of the components specified in the formulation.

### Example 3

| Component | Weight (grams) |
|---|---|
| FIRST PHASE | |
| Cetearyl Olivate + Sorbitan Olivate | 8.0 |
| Cetearyl Alcohol + Polysorbate 60 + Cetearyl Glucoside | 5.0 |
| Olive Oil PEG-7 Esters | 3.0 |
| Copernicia Cerifera Wax | 0.01 |
| Cetyl Acetate + Stearyl Acetate + Oleyl Acetate + | 0.5 |
| Acetylated lanolin Alcohols | |
| Butylated Hydroxytoluene (BHT) | 0.2 |
| Tocopheryl Acetate | 0.5 |
| Benzoic Acid | 0.2 |
| Vitis Vinifera Seed Oil | 0.5 |
| Linum Usitatissimum Seed Oil | 0.5 |
| Prunus Amygdalus Dulcis Oil | 0.5 |
| Persea Gratissima Oil | 0.5 |
| Jojoba Oil | 0.1 |
| SECOND PHASE | |
| Disodium EDTA | 0.1 |
| Propylene Glycol | 5.0 |
| Sodium Benzoate | 0.2 |
| Potassium Sorbate | 0.2 |
| Water | 70.39 |
| THIRD PHASE | |
| Water + Pentylene Glycol + Glycerin + Sodium | 2.0 |
| Lactate + Lactic Acid + Serina + Urea + Sorbitol + | |
| Sodium Chloride + Allantoin | |
| Citric Acid | 0.1 |
| Dimethyl Sulfoxide | 0.5 |
| Ethoxydiglycol diethylene glycol monoethyl ether | 1.0 |
| purified | |
| Lonicera Caprifolium extract + Lonicera japonica | 1.0 |
| extract + Water* | |
| TOTAL | 100.00 grams |

*In at least one embodiment, the mixture of lonicera caprifolium extract + lonicera japonica extract + water is neutralized to a pH of about 6.0 utilizing an appropriate amount of citric acid. In one further embodiment, the citric acid solution has a concentration of 50 percent.

The present invention is further directed to a method of preparing a nanoemulsion system for transdermal delivery of at least one pharmaceutical composition or other active agent across at least an outer layer of skin of a patient.

In accordance with at least one embodiment of the present invention, a method of preparing a nanoemulsion system for transdermal delivery of at least one pharmaceutical composition or other active agent across at least an outer layer of skin of a patient includes preparing a predetermined amount of a first phase. Examples 1 through 3 above disclose exemplary formulations which include a predetermined amount of the first phase.

The present method of preparing a nanoemulsion system for transdermal delivery of at least one pharmaceutical composition or other active agent across at least an outer layer of skin of a patient further includes preparing a predetermined amount of a second phase. As before, Examples 1 through 3 above disclose exemplary formulations which include a predetermined amount of the second phase.

The method of preparing a nanoemulsion system for transdermal delivery further includes heating the predetermined amount of the first phase to an upper threshold temperature under constant stirring. In at least one embodiment, the upper threshold temperature is in a range of about 70 degrees Celsius to about 80 degrees Celsius. In accordance with one embodiment of the present method, constant stirring is performed at a speed of about 100 revolutions per minute to about 1,000 revolutions per minute. In at least one further embodiment of the present method, constant stirring is performed at a speed of about 200 revolutions per minute.

In at least one further embodiment, the method of preparing a nanoemulsion system for transdermal delivery further includes heating the predetermined amount of the second phase to an upper threshold temperature under constant stirring. In accordance with one embodiment, the upper threshold temperature is, once again, in a range of about 70 degrees Celsius to about 80 degrees Celsius, as it is for heating the predetermined amount of the second phase. As before, in accordance with one embodiment of the present method, constant stirring is performed at a speed of about 100 revolutions per minute to about 1,000 revolutions per minute. In at least one further embodiment of the present method, constant stirring is performed at a speed of about 200 revolutions per minute.

The present method of preparing a nanoemulsion system for transdermal delivery of at least one pharmaceutical composition or other active agent across at least an outer layer of skin of a patient also includes adding the predetermined amount of the second phase at the upper threshold temperature into the predetermined amount of the first phase at the upper threshold temperature under constant stirring to form an initial mixture. Once again, in accordance with one embodiment of the present method, constant stirring is performed at a speed of about 100 revolutions per minute to about 1,000 revolutions per minute. In at least one further embodiment of the present method, constant stirring is performed at a speed of about 200 revolutions per minute.

Next, in accordance with at least one embodiment, the present method includes cooling the initial mixture to a lower threshold temperature under constant stirring. In one further embodiment, the present method includes cooling the initial mixture to a lower threshold temperature of about 40 degrees Celsius under constant stirring. As disclosed above, in accordance with one embodiment of the present method, constant stirring is performed at a speed of about 100 revolutions per minute to about 1,000 revolutions per minute. In at least one further embodiment of the present method, constant stirring is performed at a speed of about 200 revolutions per minute.

Additionally, the present method of preparing a nanoemulsion system for transdermal delivery of at least one pharmaceutical composition or other active agent across at least an outer layer of skin of a patient includes preparing a predetermined amount of a third phase. As before, Examples 1 through 3 above disclose exemplary formulations which include a predetermined amount of the third phase.

Once the predetermined amount of the third phase has been prepared, at least one embodiment of the present method includes adding the predetermined amount of the third phase to the initial mixture that has been cooled to the lower threshold temperature under constant stirring to form a final mixture. Once again, in accordance with at least one embodiment, the lower threshold temperature of about 40 degrees Celsius. Also as before, in accordance with one embodiment of the present method, constant stirring is performed at a speed of about 100 revolutions per minute to about 1,000 revolutions per minute. In at least one further embodiment of the present method, constant stirring is performed at a speed of about 200 revolutions per minute.

In accordance with at least one embodiment, the present method of preparing a nanoemulsion system for transdermal delivery of at least one pharmaceutical composition or other active agent across at least an outer layer of skin of a patient includes cooling the final mixture to a final temperature under constant stirring to form the nanoemulsion system. In accordance with at least one further embodiment, the present method includes cooling the final mixture to a final temperature of about 25 degrees Celsius under constant stirring to form the nanoemulsion system. In accordance with one embodiment of the present method, and as disclosed hereinabove, constant stirring is performed at a speed of about 100 revolutions per minute to about 1,000 revolutions per minute. In at least one further embodiment of the present method, constant stirring is performed at a speed of about 200 revolutions per minute

Once the nanoemulsion system has been formed, at least one embodiment of the present method includes adjusting a pH of the nanoemulsion system, and in at least one further embodiment, the present method includes adjusting a pH of the nanoemulsion system to a pH of about 5 to a pH of about 7.

As a final matter, in at least one embodiment, the present method includes storing the pH adjusted nanoemulsion system in an appropriate storage container. In at least one embodiment, the pH adjusted nanoemulsion system has a biocompatible Zeta Potential of about −40 millivolts to about −60 millivolts, and in one further embodiment, the pH adjusted nanoemulsion system has a biocompatible Zeta Potential of about −42 millivolts to about −54 millivolts.

Once a nanoemulsion system has been prepared in accordance with the method of the present invention, a pharmaceutical composition or other active agent is prepared for incorporation into the nanoemulsion system. In at least one embodiment, when the pharmaceutical composition or other active agent comprises a solid phase, a knife mill is utilized to reduce the size of the pharmaceutical composition or other active agent. In at least one embodiment a knife mill is operated at a speed of about 20,000 revolutions per minute, and in one further embodiment, the pharmaceutical composition or other active agent is processed in the knife mill for three cycles of about one minute each at 20,000 revolutions per minute.

After the pharmaceutical composition or other active agent has been prepared for incorporation into a nanoemulsion system as described above, if necessary, Example 4 below is exemplary of a formation for 100.00 grams of a nanoemulsion system for transdermal delivery of at least one pharmaceutical composition or other active agent, i.e., progesterone, across at least an outer layer of skin of a patient, including the weight, in grams, of each of the components specified in the formulation. More in particular, Example 4 represents a formulation for the preparation of 100 one gram doses of a nanoemulsion system for transdermal delivery of progesterone across at least an outer layer of skin of a patient, wherein each dose comprises 50 milligrams of progesterone.

### Example 4

| Component | Weight (grams) |
|---|---|
| Progesterone | 5.0 |
| Ethoxydiglycol diethylene | 5.0 |
| glycol monoethyl ether purified | |
| Olea Europaea Fruit Oil | 1.0 |
| Nanoemulsion | 89.0 |
| TOTAL | 100.0 grams |

NOTE:
The at least one pharmaceutical composition or other active agent, i.e., progesterone, is dispersed in the solvents under constant stirring at a speed of about 200 revolutions per minute for a period of time of about 10 minutes.

In at least one further embodiment of the present invention, after at least one pharmaceutical composition or other active agent is incorporated into a nanoemulsion system prepared in accordance with the present invention, the nanoemulsion system comprising the at least one pharmaceutical composition or other active agent is subjected to particle size reduction and standardization. More in particular, in at least one embodiment, the nanoemulsion system comprising the at least one pharmaceutical composition or other active agent is processed through a roll mill operating at about 700 revolutions per minute. In at least one further embodiment, the nanoemulsion system comprising the at least one pharmaceutical composition or other active agent is processed three consecutive times through a roll mill operating at about 700 revolutions per minute to produce a final mixture having a narrow particle size distribution of about 25 nanometers to about 75 nanometers and about 100 nanometers to about 250 nanometers. In still one further embodiment, the nanoemulsion system comprising the at least one pharmaceutical composition or other active agent is processed three consecutive times through a roll mill operating at about 700 revolutions per minute to produce a final mixture having a narrow particle size distribution of about 35 nanometers to about 66 nanometers and about 108 nanometers to about 234 nanometers.

A nanoemulsion system comprising at least one pharmaceutical composition or other active agent prepared in accordance with the present invention may be utilized by a patient in the following manner. First, the patient, or whomever is going to apply the nanoemulsion system to the patient, must thoroughly wash his or her hands so as to avoid transport of any contaminants across the outer layer of the skin. A predetermined dosage amount of the nanoemulsion system comprising at least one pharmaceutical composition or other active agent rubbed gently on an area of clean skin of the patient, preferably, in areas with little hair as possible, such as, an inner face of an upper arm or thigh. After the nanoemulsion system comprising at least one pharmaceutical composition or other active agent has been substantially absorbed through the outer layer of skin of the patient, rubbing is discontinued for a period of about ten seconds. Finally, rubbing is resumed until the nanoemulsion system comprising at least one pharmaceutical composition or other active agent has been completely absorbed through the outer layer of skin of the patient. The hands of the patient of other person applying the nanoemulsion system comprising at least one pharmaceutical composition or other active agent should be thoroughly washed once again after application to the patient. However, the application site on the skin of the patient should not be washed or otherwise exposed to water, moisture, or other agents which may be absorbed through the skin for a period of at least about 60 minutes after application.

Since many modifications, variations and changes in detail can be made to the described embodiments of the invention, it is intended that all matters in the foregoing description be interpreted as illustrative and not in a limiting sense. Thus, the scope of the invention should be determined by the appended claims and their legal equivalents.

What is claimed is:

1. A nanoemulsion system for transdermal delivery of at least one pharmaceutical composition or other active agent across at least an outer layer of skin of a patient, said nanoemulsion system comprising:
a first phase,
a second phase,
a third phase, and
the at least one pharmaceutical composition or other active agent.

2. The nanoemulsion system as recited in claim 1 wherein said first phase comprises an oil phase.

3. The nanoemulsion as system recited in claim 2 wherein said first phase comprises an amount of at least one of cetearyl olivate, sorbitan olivate, cetearyl alcohol, polysorbate 60, cetearyl glucoside or olive oil PEG-7 esters.

4. The nanoemulsion system as recited in claim 1 wherein said second phase comprises an aqueous phase.

5. The nanoemulsion system as recited in claim 4 wherein said second phase comprises an amount of water.

6. The nanoemulsion system as recited in claim 4 wherein said second phase comprises an amount of at least one of propylene glycol, disodium EDTA, sodium benzoate or potassium sorbate.

7. The nanoemulsion system as recited in claim 1 wherein said third phase comprises a final phase.

8. The nanoemulsion system as recited in claim 7 wherein said final phase comprises an amount of at least one of a preservation agent or a permeation promoter.

9. The nanoemulsion system as recited in claim 7 wherein said final phase comprises an amount of at least one of ethoxydiglycol diethylene glycol monoethyl ether or dimethyl sulfoxide.

10. The nanoemulsion system as recited in claim 7 wherein said final phase comprises an amount of a natural preservative.

11. The nanoemulsion system as recited in claim 10 wherein said natural preservative comprises an amount of at least one of Lonicera caprifolium extract or Lonicera japonica extract in an amount of water.

12. A method of preparing a nanoemulsion system for transdermal delivery of at least one pharmaceutical composition or other active agent across at least an outer layer of skin of a patient, said method comprising:
preparing a predetermined amount of a first phase,
preparing a predetermined amount of a second phase,
heating the predetermined amount of the first phase to an upper threshold temperature under constant stirring,
heating the predetermined amount of second phase to the upper threshold temperature under constant stirring,
adding the predetermined amount of second phase at the upper threshold temperature into the predetermined amount of the first phase at the upper threshold temperature under constant stirring to form an initial mixture,
cooling the initial mixture to a lower threshold temperature under constant stirring,
preparing a predetermined amount of a third phase,
adding the predetermined amount of the third phase to the initial mixture cooled to the lower threshold temperature under constant stirring to form a final mixture,
cooling the final mixture to a final temperature under constant stirring to form the nanoemulsion system, and
adjusting a pH of the nanoemulsion system.

13. The method as recited in claim 12 further comprising heating the predetermined amount of the first phase to an upper threshold temperature of about 70 degrees Celsius to about 80 degrees Celsius under constant stirring.

14. The method as recited in claim 12 further comprising heating the predetermined amount of the second phase to an upper threshold temperature of about 70 degrees Celsius to about 80 degrees Celsius under constant stirring.

15. The method as recited in claim 12 further comprising cooling the initial mixture to a lower threshold temperature of about 40 degrees Celsius under constant stirring.

16. The method as recited in claim 12 further comprising adding the predetermined amount of the third phase to the initial mixture cooled to a lower threshold temperature of about 40 degrees Celsius under constant stirring to form the final mixture.

17. The method as recited in claim 12 further comprising cooling the final mixture to a final temperature of about 25 degrees Celsius under constant stirring to form the nanoemulsion system.

18. The method as recited in claim 12 further comprising adjusting the pH of the nanoemulsion system to about 5 to a pH of about 7.

19. The method as recited in claim 12 wherein constant stirring is performed at a speed of about 100 revolutions per minute to about 1,000 revolutions per minute.

20. The method as recited in claim 12 wherein constant stirring is performed at a speed of about 200 revolutions per minute.
